(19) United States
(12) Patent Application Publication
(10) Pub. No.: US 2011/0014181 A1
(43) Pub. Date: Jan. 20, 2011

(54) Microneedle Delivery Device and Methods of Using Same

(75) Inventor: RONAN THORNTON, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/505,939

(22) Filed: Jul. 20, 2009

(51) Int. Cl. A61M 5/00; A61K 31/366; A61K 33/00; A61K 39/395; A61K 31/7088; A61K 31/436; A61K 35/00

(52) U.S. Cl. 424/130.1; 514/460; 424/718; 514/44 A; 514/44 R; 514/291; 424/93.7; 604/173; 604/272

(57) ABSTRACT

Described herein are microneedle bioactive agent delivery systems, associated apparatus and methods of using such. The microneedles described herein are deliverable using a needle or syringe apparatus that can interface with existing medical devices or the devices can be used as standalone systems. The systems deliver at least one bioactive agent to a tissue in need thereof, for example, the myocardium.

### FIELD OF THE INVENTION

The present invention generally relates to microneedle apparatus and systems and methods of injecting of bioactive agents into tissues in need thereof.

### BACKGROUND

Heart disease, including myocardial infarction (MI), is a leading cause of death and disability in human beings, particularly in the western world, most particularly among males. Myocardial infarction can result in an acute depression in ventricular function and expansion of the infarcted tissue under stress. This triggers a cascading sequence of myocellular events known as remodeling. In many cases, this progressive myocardial infarct expansion and remodeling leads to deterioration in ventricular function and heart failure. Such ischemic cardiomyopathy is the leading cause of heart failure in the United States.

According to the American Heart Association, in the year 2000 approximately 1,100,000 new myocardial infarctions occurred in the United States. For 650,000 patients this was their first myocardial infarction, while for the other 450,000 patients this was a recurrent event. Two hundred-twenty thousand people suffering Ml die before reaching the hospital. Within one year of the myocardial infarction, 25% of men and 38% of women die. Within 6 years, 22% of men and 46% of women develop heart failure, of which 67% are disabled. This is despite modern medical therapy.

The consequences of myocardial infarction are often severe and disabling. When a myocardial infarction occurs, the myocardial tissue that is no longer receiving adequate blood flow dies and is replaced with scar tissue. This infarcted tissue cannot contract during systole, and may actually undergo lengthening in systole and leads to an immediate depression in ventricular function. This abnormal motion of the infarcted tissue can cause delayed or abnormal conduction of electrical activity to the still surviving peri-infarct tissue (tissue at the junction between the normal tissue and the infarcted tissue) and also places extra structural stress on the peri-infarct tissue.

Treatments following myocardial infarction that are used currently, and that have been used in the past, are varied. Immediately after a myocardial infarction, preventing and treating ventricular fibrillation and stabilizing the hemodynamics are well-established therapies. However, regeneration of the damaged or injured tissues or stem cell therapy have become popular methods of treating cardiac tissue after conditions such as myocardial infarction.

The direct or selective delivery of agents to cardiac tissue is often preferred over the systemic delivery of such agents for several reasons. One reason is the substantial expense and small amount of the medical agents available, for example, agents used for gene therapy. Another reason is the substantially greater concentration of such agents that can be delivered directly into cardiac tissue, compared with the dilute concentrations possible through systemic delivery. Yet another reason is that systemic administration is associated with systemic toxicity at doses required to achieve desired drug concentrations in the cardiac tissue.

One mode of delivering medical agents to cardiac tissue is by epicardial, direct injection into cardiac tissue during an open chest procedure. Another approach taken to deliver medical agents into cardiac tissue has been an intravascular approach. Catheters may be advanced through the vasculature and into the heart to inject materials into cardiac tissue from within the heart. Another approach is to deliver materials into cardiac wall from within the chamber of the heart, an endocardial approach. Furthermore, additional therapies being developed for treating injured cardiac tissue include the injection of cells and/or other biologic agents into ischemic cardiac tissue or placement of cells and/or agents onto the ischemic tissue. One therapy for treating infarcted cardiac tissue includes the delivery of cells that are capable of maturing into actively contracting cardiac muscle cells or regenerating cardiac tissue. Examples of such cells include myocytes, myoblasts, mesenchymal stem cells, and pluripotent cells. Delivery of such cells into cardiac tissue is believed to be beneficial, particularly to prevent or treat heart failure.

It has been postulated that after acute or chronic injury to the heart, endogenous regenerative cells attempt to restore some or all function to the injured tissue. The present description provides systems and methods to deliver such cells to cardiac tissue.

### SUMMARY

Described herein are microneedle bioactive agent delivery systems, associated apparatus and methods of using such. The microneedles described herein are deliverable using a needle or syringe apparatus that can interface with existing medical devices or the devices can be used as standalone systems. The systems deliver at least one bioactive agent to a tissue in need thereof, for example, cardiac tissue.

In one embodiment, microneedle drug delivery devices are described comprising: a needle comprising a body, a proximal end and a distal end, wherein the distal end is closed and the proximal end is attached to a delivery means, and the body has at least one channel traversing interior of the body from the distal end to the proximal end, the at least one channel terminates at an exit port at the distal end of the needle; and at least one microneedle housed within at least one channel for delivery through the exit port of at least one channel.

Further described herein, in another embodiment, are methods for delivering a microneedle comprising: a) selecting a patient who is a candidate for local delivery of at least one bioactive agent to a tissue in need thereof; b) providing the microneedle injection device as described herein; c) inserting the needle into the tissue; d) advancing at least one microneedle out of at least one channel through the exit port and into the tissue; e) retracting the needle from the tissue; and f) delivering one or more bioactive agents to the tissue using the microneedle.

In one embodiment, the methods further comprise the step of retracting the at least one microneedle back through the exit port into at least one channel. In another embodiment of the methods, eight microneedles are injected in a radial pattern into the tissue substantially perpendicular to at least one channel of the needle.

In other embodiments, the delivery body has four channels or eight channels from which microneedles can be injected into a tissue. In some embodiments, at least one microneedle is injected into the tissue substantially perpendicular to the needle from at least one channel of the needle.

The at least one microneedle can be degradable or can be non-biodegradable. If biodegradable, the microneedle can be made of a biodegradable polymer and a bioactive agent can be dispersed therein. Or, in other embodiments, the microneedle can be made of metal.

Bioactive agents utilized in the devices, systems and methods described herein, in one embodiment, are selected from the group consisting of anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) zotarolimus (ABT-578), somatic cells, stem cells and combinations thereof.

Further still, in one embodiment described herein are microneedle drug delivery devices comprising: a needle comprising a body, a proximal end and a distal end, wherein the distal end is closed and the proximal end is attached to a delivery means, and the body has eight channels traversing the interior of the body from the distal end to the proximal end, the eight channels terminate at eight exit ports at the distal end of the needle; and eight microneedles comprising at least one bioactive agent, each of the eight microneedles housed within one of the eight channels for delivery through the eight exit ports of the eight channels.

### BRIEF DESCRIPTION OF THE DRAWINGS

FIG. 1A-C illustrate an example embodiment of a microneedle delivery system. FIG. 1A illustrates a microneedle outside a delivery system as described herein. FIG. 1B illustrates microneedles within the system. FIG. 1C illustrates an example embodiment, wherein a cross-section of a needle contains eight channels and eight microneedles.

FIGS. 2A-D illustrate an example embodiment of a microneedle delivery system with retractable microneedles.

FIGS. 3A-C illustrate an example embodiment of a microneedle delivery system with non-retractable biodegradable microneedles.

FIG. 4 illustrates an example microneedle delivery configuration.

FIGS. 5A-C illustrate a microneedle with a biodegradable tube for injection.

FIGS. 6A-C illustrate a microneedle syringe system for bioactive agent delivery.

FIG. 7 schematically illustrates an epicardial approach to delivery using a catheter as described herein.

FIG. 8A-B schematically illustrates an endocardial approach to delivery using a catheter as described herein. FIG. 8A illustrates an anterograde endocardial approach through the venous system and FIG. 8B illustrates a retrograde endocardial approach through the arterial system.

FIG. 9A-B schematically illustrates a transvascular approach to delivery using a catheter as described herein. FIG. 9A depicts a venous approach and FIG. 9B depicts an arterial approach through the coronary artery.

FIG. 10 illustrates a needle being injected substantially perpendicular to cardiac tissue using the systems and methods as described herein.

### DEFINITION OF TERMS

Certain terms as used in the specification are intended to refer to the following definitions, as detailed below. Where the definition of terms departs from the commonly used meaning of the term, applicant intends to utilize the definitions provided below, unless specifically indicated.

As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

As used herein “biodegradable” refers to a polymeric composition that is biocompatible and subject to being broken down in vivo through the action of normal biochemical pathways. From time-to-time bioresorbable and biodegradable may be used interchangeably, however, they are not coextensive. Biodegradable polymers may or may not be reabsorbed into surrounding tissues, however, all bioresorbable polymers are considered biodegradable. Biodegradable polymers are capable of being cleaved into biocompatible byproducts through chemical- or enzyme-catalyzed hydrolysis.

As used herein, “cardiac tissue injury” refers to any area of abnormal tissue in the heart caused by a disease, disorder or injury and includes damage to the epicardium, endocardium, and/or myocardium. Non-limiting examples of causes of cardiac tissue injury include acute or chronic stress (systemic hypertension, pulmonary hypertension, valve dysfunction, etc.), coronary artery disease, ischemia or infarction, inflammatory disease and cardiomyopathies. Cardiac tissue injury most often involves injury to the myocardium and therefore, for the purposes of this disclosure, myocardial injury is equivalent to cardiac tissue injury. Furthermore, there are occasions when the injury is acute, such as in an acute myocardial infarction, where the injury may be referred to as an injurious event. Injured cardiac tissue includes tissue that is ischemic, infarcted, or otherwise focally or diffusely diseased. Cardiac tissue injury also includes acute trauma resulting from external means such as, but not limited to, blunt force or puncture.

As used herein “nonbiodegradable” refers to compositions and materials that are biocompatible and not subject to being broken down in vivo through the action of normal biochemical pathways.

As used herein, the term “percutaneous” refers to any penetration through the skin of the patient, whether in the form of a small cut, incision, hole, cannula, tubular access sleeve or port or the like. A percutaneous penetration may be made in an interstitial space between the ribs of the patient or it may be made elsewhere, such as the groin area of a patient.

As used herein “pharmaceutically acceptable” refers to all derivatives and salts that are not substantially toxic at effective levels in vivo.

As used herein “substantially perpendicular” refers to an angle that is about perpendicular to or about 90 degrees relative to. For example, substantially perpendicular can mean within about 5%, about 10% or even about 20% of being perpendicular.

### DETAILED DESCRIPTION

Described herein are microneedle bioactive agent delivery systems, associated apparatus and methods of using such. The microneedles described herein are deliverable using a needle or syringe apparatus that can interface with existing medical devices or the devices can be used as standalone systems. The systems deliver at least one bioactive agent to a tissue in need thereof.

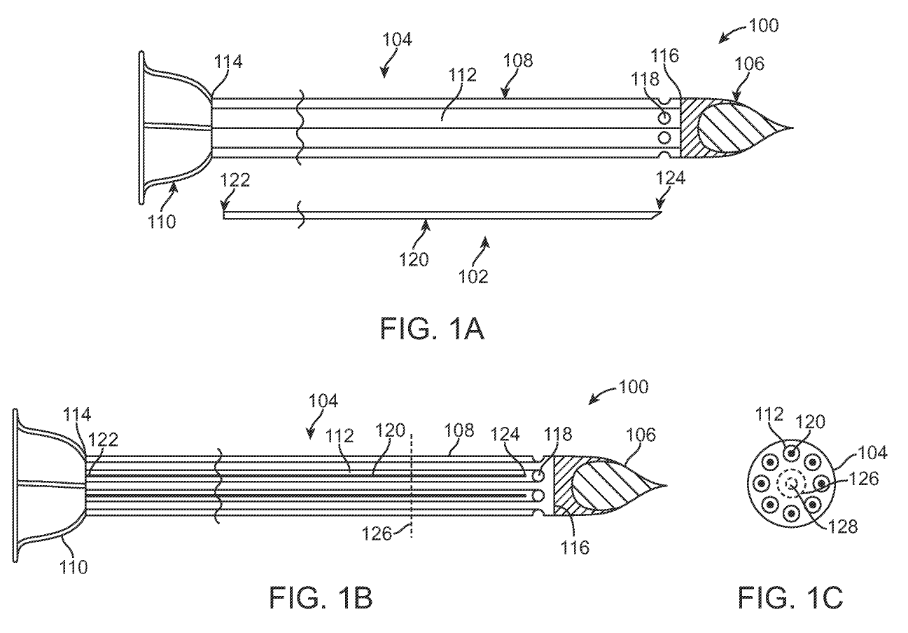

Referring to FIGS. 1A-1C, needle system 100 illustrates an example embodiment according to the present description. Needle system 100 includes at least one microneedle 102 and needle 104. Needle 104 can be made from any needle commonly known in the art and includes bevel 106, body 108 and optionally lock tip 110. Lock tip 110 can be replaced by any connector used to connect needles to a delivery means, for example, screw connectors, twist-and-lock connectors, compression fittings, and the like. A delivery means can be a device such as, but not limited to, a syringe, a delivery pump, a catheter, and the like. In some example embodiments, lock tip 110 need not be used if an entire needle system is used or a catheter system is used.

Needle 104 can be manufactured or formed as a modified version of a standard needle or can be manufactured directly. Bevel 106 is closed-off or sealed, isolating the interior of body 108 from bevel 106 (through which bioactive agents are normally delivered). The sealing of bevel 106 can be accomplished by any means known in the art, for example, by soldering or simply machining needle 104 in such a configuration during manufacturing. Needle 104, for example, is sealed off so that blood cannot pass through bevel 106 into body 108. Rather, bevel 106 can be solid impenetrable mass.

Needle 104 is preferably between about 16 gauge and about 2 gauge, preferably about 8 gauge. The length of needle 104 is dependent on the tissue being treated or microneedle 102 being used. However, generally, needle 104 is between about 1 inch and about 5 inches, preferably about 2 inches.

Needle 104 includes body 108 which is hollow and configured to deliver at least one microneedle 102. Body 108 includes at least one channel 112 traversing the interior from proximal end 114 to distal end 116. Channel 112 houses microneedle 102 for delivery. Exit port 118 terminates each channel 112 and provides an outlet through which microneedle 102 is ejected from needle 104.

In some example embodiments, channel 112 is gradually bent at distal end 116 so that microneedle 102 traverses exit port 118 about perpendicular to (about 90 degrees relative to or substantially perpendicular) channel 112. Bevel 106 is sealed such that microneedle 102 cannot be ejected out of needle 104 through distal end 116, but rather through exit port 118. As such, channel 112 can be configured to deliver microneedle 102 at any angle from about 1 degree to about 179 degrees preferably by a bend in the proximal end of each channel 112. For example, the proximal end of a channel can be curved 90 degrees in order to inject a microneedle perpendicularly, or substantially so, out of needle 104.

Microneedle 102 can be manufactured from non-biodegradable materials such as metals. These metals can include, but are not limited to stainless steel, tantalum, titanium, iron, aluminum, Nickel-Titanium alloys, shape memory alloys, super elastic alloys, low-modulus Ti—Nb—Zr alloys, colbalt-nickel alloy steel (MP-35N) and the like. Non-biodegradable polymers can also be used. Non-biodegradable polymers include polymers with a relatively low chronic tissue response such as polyurethanes, silicones, and polyesters could be used and other polymers could also be such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; carboxymethyl cellulose, and combinations thereof. In one example embodiment, microneedle 102 is manufactured from poly methyl methacrylate (PMMA). In another example embodiment, microneedle 102 is manufactured from polycarbonate.

Microneedle 102, when manufactured from a non-biodegradable material, for example, can be hollow with generally cylindrical body 120 with blunt end 122 and beveled end 124. Microneedle 102 is shorter in length than channel 112. In one example embodiment, microneedle 102 is preferably short enough to fit in channel 112 without projecting into the section of channel 112 wherein bending begins. In some embodiments however, microneedle 102 extends beyond the bending section and terminated just before exit port 118. Microneedle 102 has a diameter that is small enough to fit within channel 112 and allow ejection out of exit port 118.

At least one bioactive agent can be housed within cylindrical body 120 of microneedle 102 in any deliverable form known in the art. For example, bioactive agents can be in liquid or solid form or can be in the form of a gel or polymer. Bioactive agents useful herein include anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) zotarolimus (ABT-578), somatic cells, stem cells and combinations thereof.

The stem cells useful according to the present description include cells that proliferate and engraft into the myocardium of the patient and a physiologic carrier solution. The cells may be derived from a single individual or multiple individuals and may be of the same species or a different species than the recipient. In one embodiment, the cells are autologous.

Sources of cells suitable for use in cell preparation can include, but are not limited to, embryonic, fetal, post-natal or adult stem or progenitor cells, cardiomyocytes, skeletal myocytes, skeletal myoblasts, mesenchymal stem cells, endothelial progenitor cells, hematological cells, immune cells, and combinations thereof. Sources of stem cells include, but are not limited to, bone marrow, blood, adipose tissue, gonads, skeletal or cardiac muscle, or any tissue containing stem cells. The cells may be obtained by any suitable method as would be known to persons of ordinary skill in the art.

Whether the microneedles are degradable or nondegradable, microneedle 102 has an inner diameter of between about 25 μm and about 200 μm. In one example embodiment, microneedle 102 has an inner diameter between about 30 μm and about 100 μm, or about 50 μm. Microneedles with an inner diameter of about 50 μm allow for the delivery of very small amounts of stem cells, typically only about one or two cells thick. Such a configuration of stem cells allows more cell surface area to see surrounding tissue as opposed to pools of stem cells wherein each stem cell more likely than not only sees adjacent stem cells. Without wishing to be bound to or by any particular theory, it is believed that it is this phenomenon that increased the therapeutic effect of the methods described herein.

Microneedle 102 can also be manufactured as a biodegradable structure that has either a solid or hollow generally cylindrical body 120 with blunt end 122 and beveled end 124. Biodegradable materials such as biodegradable metals include alloys of magnesium with other metals including, but not limited to, aluminum and zinc. In one embodiment, the magnesium alloy comprises between about 1% and about 10% aluminum and between about 0.5% and about 5% zinc.

Further, microneedles that are biodegradable can be manufactured of bioabsorbable polymers selected from poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen, hyaluronic acid and combinations thereof.

One or more bioactive agents can be associated with the biodegradable material whether it be metal, polymer or a combination thereof. Associated with, as used herein, includes dispersed within, implanted within, formed with, coated on or any combination thereof. Bioactive agents can also be associated with only certain portions of microneedle 102. For example, a bioactive agent can be coated on the proximal and distal end of a microneedle.

Microneedle 102 can further be manufactured from a porous biodegradable material. Porous polymers can be formed using leachable materials such as salts, for example, sodium chloride. The polymer is formed including a salt of a given diameter, and once formed, the salt can be leached from the polymer leaving pores of a given diameter based on the size of the salt. Once formed, the pores can be loaded with bioactive agents by methods known in the art.

As illustrated in FIG. 1C, needle 104 can have at least one central bored channel 126. The purpose of at least one central bore 126 is to deliver one or more substances through at least a portion of bevel 106. Although bevel 106 is sealed, it can have one or more destructible seals (not shown) whereby a substance can be ejected therethrough. For example, a needle 128 can be housed within a central bore and contain a polymerizable substance to seal the hole when needle 104 is removed from the tissue. In another example embodiment, a bioactive agent can be delivered as needle 104 is removed from the tissue. At least one central bore 126 does not interfere with the delivery of microneedle 102 and is a completely different channel/bore.

Methods of delivering microneedles according to the present description are dependent on whether the microneedle is biodegradable or non-biodegradable. The first step in delivering bioactive agents as described herein is to select a patient who is a candidate for local delivery of at least one bioactive agent to a tissue in need thereof. After an appropriate patient has been identified, the bioactive agents can be delivered.

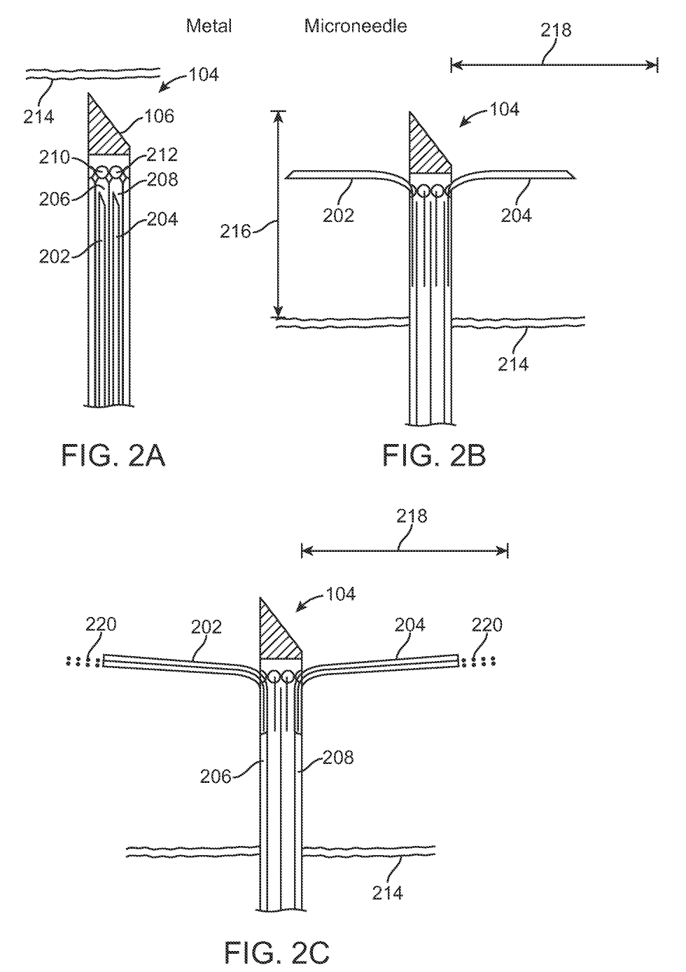

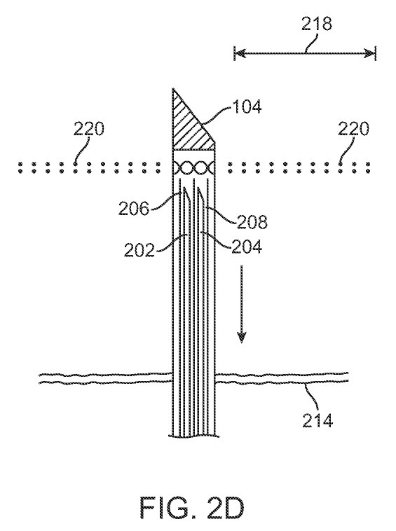

Non-biodegradable microneedles are delivered as generally illustrated in FIGS. 2A-D. In FIG. 2A, needle 104 comprises first microneedle 202 housed in first chamber 206 and second microneedle 204 housed in second chamber 208. First chamber 206 and second chamber 208 include first exit port 210 and second exit port 212. Both exit ports are optionally fitted with a polymeric septum (not shown) to keep first microneedle 202 and second microneedle 204 sealed within their respective chambers (206, 208) until injection. For injection, first, needle 104 is advanced into tissue 214 via bevel 106.

As illustrated in FIG. 2B, once needle 104 has been advanced into tissue 214 to appropriate depth 216, first microneedle 202 and second microneedle 204 are advanced out of first exit port 210 and second exit port 212 into tissue 214, perpendicular to needle 104. Then, as illustrated in FIG. 2C, after first microneedle 202 and second microneedle 204 have been advanced out of needle 104 to appropriate distance 218, first microneedle 202 and second microneedle 204 can slowly be retracted back into first chamber 206 and second chamber 208 while slowly allowing bioactive agent 220 to be left in the trails of the microneedles.

After first microneedle 202 and second microneedle 204 have been fully retracted back into first chamber 206 and second chamber 208, needle 104 can be retracted out of tissue 214 (FIG. 2D). Bioactive agent 220 is left in tissue 214 in at least one trail configuration.

Injection out of and/or retraction back into needle 104 of one or more microneedles can be accomplished by any means known in the art. For example, aspiration, pressure, force, a physical connection to a plunger, an indirect connection to a plunger, a mechanical means, or the like can be used to move microneedles out of or into needle 104. The same means can be used to deposit a composition, such as a bioactive agent, out of a microneedle into a tissue.

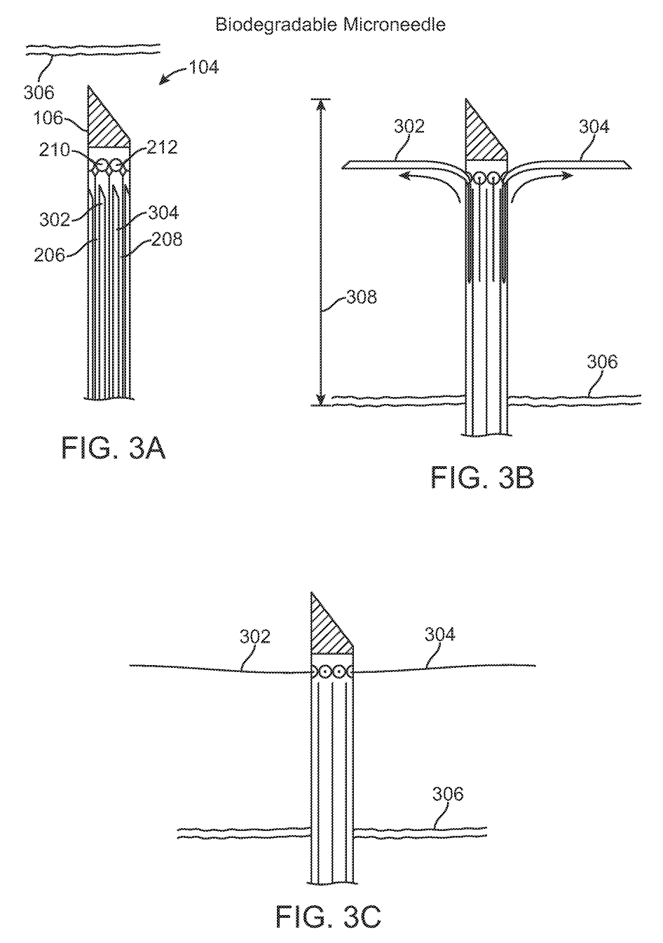

Biodegradable microneedles which are not to be retracted back into needle 104 but are to remain within the injected tissue are delivered as generally illustrated in FIGS. 3A-C. In FIG. 3A, needle 104 comprises first biodegradable microneedle 302 housed in first chamber 206 and second biodegradable microneedle 304 housed in second chamber 208. First chamber 206 and second chamber 208 include first exit port 210 and second exit port 212. Both exit ports are optionally fitted with a polymeric septum (not shown) to keep first biodegradable microneedle 302 and second biodegradable microneedle 304 sealed within their respective chambers (206, 208) until injection. For injection, first, needle 104 is advanced into tissue 306 via bevel 106.

As illustrated in FIG. 3B, once needle 104 has been advanced into tissue 306 to appropriate depth 308, first biodegradable microneedle 302 and second biodegradable microneedle 304 are advanced out of first exit port 210 and second exit port 212 into tissue 306, perpendicular to needle 104. As illustrated in FIG. 3C, once first biodegradable microneedle 302 and second biodegradable microneedle 304 are fully advanced out of their respective chambers, they are left in the tissue and needle 104 is retracted from tissue 306. The result is first biodegradable microneedle 302 and second biodegradable microneedle 304 left in tissue 214.

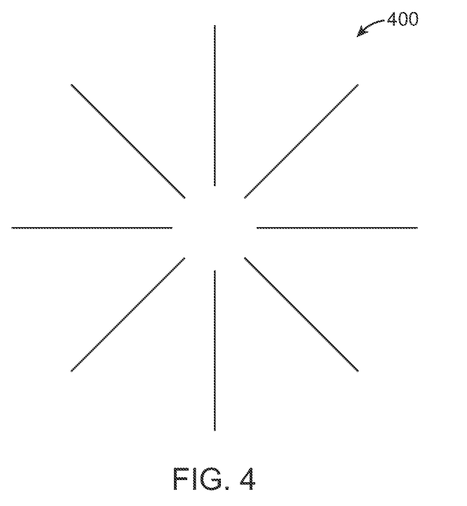

For the widest local bioactive agent delivery, bioactive agents are delivered in a planar fashion with bioactive agents at the most distant positions from each other. For example, FIG. 4 illustrates a planar configuration wherein eight biodegradable microneedles are injected into a tissue resulting in configuration 400.

More elaborate configurations are also within the scope of the present description. For example, three-dimensional configurations are attainable. For example, if 12 biodegradable microneedles are injected into a tissue at alternating angles of 45 degrees, 90 degrees (perpendicular to needle 104) and 135 degrees, a semi-spherical injection pattern is achieved. Table 1 includes several example embodiments of 3-dimensional configurations that can be achieved using the systems and apparatus of the present disclosure.

In alternative embodiments, the disclosed systems comprise from one microneedle to 12 or more microneedles. Even 24 or 36 microneedles can be used. The limiting factor for number of microneedles becomes the size of the needle used to inject them. As long as the skilled artisan is able to safely inject a particular number of microneedles, it is though to be within the scope of the present disclosure.

TABLE 1

| | Vertical angle between | Horizontal angle |
|---|---|---|
| Number of | microneedles and | between |
| microneedles | needle | microneedles |
| 2 | all 90 | all 180 |
| 3 | all 90 | all 120 |
| 4 | all 90 | all 90 |
| 5 | all 90 | all 72 |
| 6 | all 90 | all 60 |
| 9 | all 90 | all 40 |
| 9 | (45, 90, 135) × 3 | each set 120 |
| 12 | all 90 | all 30 |
| 12 | (45, 90, 135) × 4 | each set 90 |

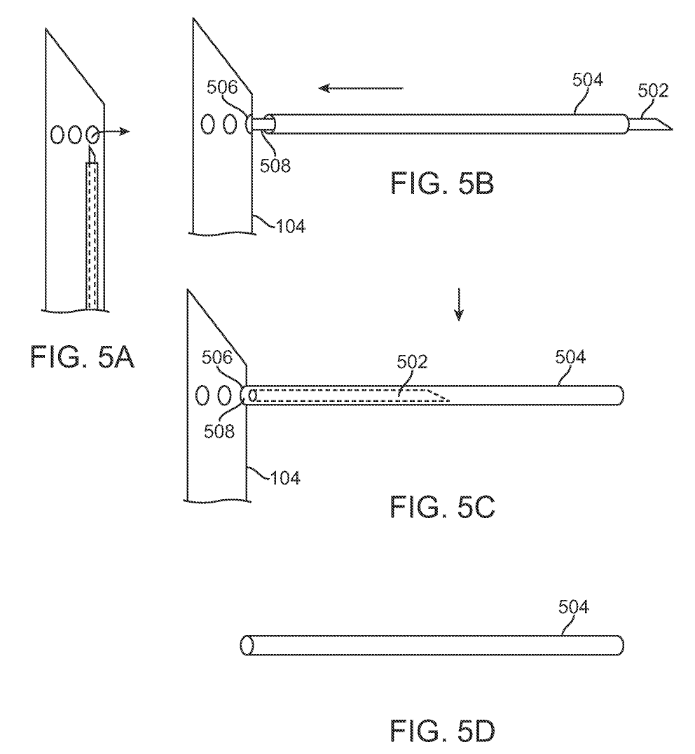

In one example embodiment, a biocompatible cylindrical polymer containing at least one bioactive agent can be delivered using a non-biodegradable metal microneedle. FIG. 5 illustrates such a method. Non-biodegradable microneedle 502 including polymeric cylinder 504 containing a bioactive agent is advanced through exit port 506 and membrane 508 of needle 104. As non-biodegradable microneedle 502 is retracted back into needle 104, membrane 508 pushes polymeric cylinder 504 off of non-biodegradable microneedle 502 and is left in the tissue. Polymeric cylinder 504 degrades over time and the increased surface area compared to a solid biodegradable microneedle helps enhance bioactive agent delivery to the surrounding tissues.

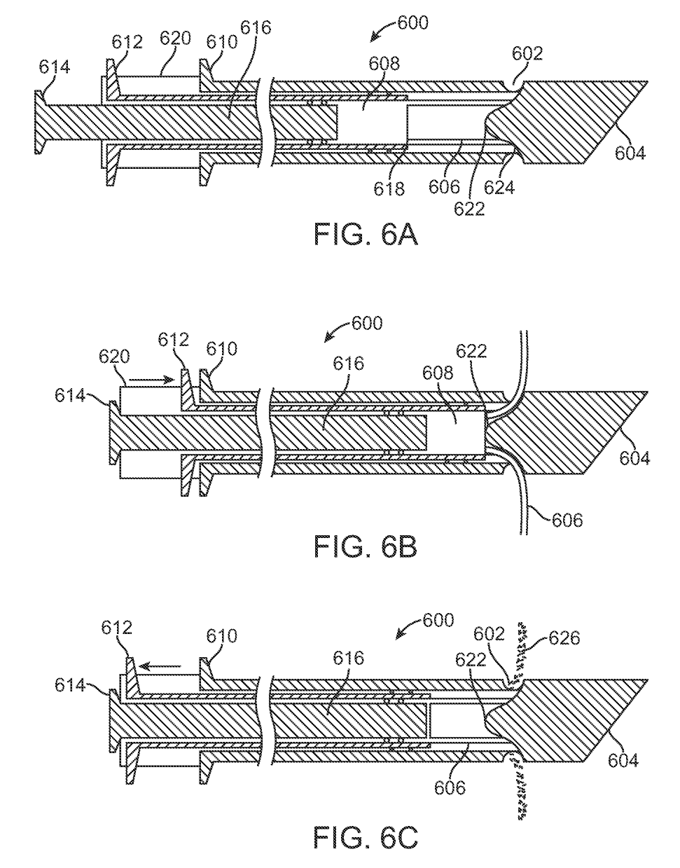

Another example embodiment is illustrated in FIG. 6. System 600 includes at least one exit port 602, closed end needle 604, at least one microneedle 606, fluid chamber 608, outer handle 610, middle handle 612, inner handle 614 and plunger 616. As illustrated in FIG. 6A, system 600 is ready to use once an optional cover (not shown) is removed from closed end needle 604. At least one microneedle 606 is pre-filled with stem cells and/or another bioactive agent or bioactive agent composition. If stem cells are used, it can be advantageous to keep the microneedles cold or load the stem cells into the microneedles just prior to injection. Each microneedle 606 is secured to fluid chamber 608 at connection point 618. In one example embodiment, system 600 is fitted with plunger guide 620. Plunger guide 620 allows, in some circumstances, for easier operability of system 600, by preventing an operator from over-injecting microneedle 606.

Closed end needle 604 of system 600, as illustrated in FIG. 6B, is injected into the tissue of a patient and then an operator depresses middle handle 612 thereby advancing at least one microneedle 606 out of at least one exit port 602 and into the surrounding tissue. Middle handle 612 is advanced until fluid chamber 608 comes into contact with stop 622. At that point, at least one microneedle 606 is fully advanced out of system 600 at an angle determined by at least one channel guide 624. If system 600 incorporates plunger guide 620, pressure can be applied to inner handle 614 until it strikes and is stopped at the top of plunger guide 620, a point at which at least one microneedle 606 is fully advanced out of system 600. Again, this prevents over-injecting of microneedle 606 and allows for a precise injection depth.

Then, as illustrated in FIG. 6C, while holding inner handle 614 stationary, middle handle 612 is regressed back toward inner handle 614. This process retracts at least one microneedle 606 back into system 600 while at the same time advancing the fluid out of fluid chamber 608 into at least one microneedle 606. This advancement of fluid causes contents 626 of at least one microneedle 606 to be displaced into the tissue as at least one microneedle 606 is retracted. Once at least one microneedle 606 is fully retracted, syringe system 600 can be removed from the patient. If system 600 incorporates plunger guide 620, pressure can be applied to inner handle 614 for example with the thumb and middle handle retraced for example with the pointer finger and middle finger until middle handle comes in contact with plunger guide 620 wherein at least one microneedle 606 is fully retracted.

Tissues that can be treated using the systems and associated apparatus disclosed herein include, but are not limited to, muscles, skin and spinal tissue. In some example embodiments, cardiac tissue, more specifically, the myocardium is treated using the present systems and methods.

In order to deliver one or more bioactive agents from, or within, one or more microneedles to a target site within the myocardium, a clinician may use one of a variety of access techniques. These include surgical (sternotomy, thoracotomy, mini-thoracotomy, sub-xiphoid) approaches and percutaneous (transvascular and endocardial) approaches. Once access has been obtained, the composition may be delivered via epicardial, endocardial, or transvascular approaches. The microneedles may be delivered to the cardiac wall tissue in one or more locations. This includes intra-myocardial, sub-endocardial, and/or sub-epicardial administration.

One method to predictably deliver microneedles into such a moving target tissue is to time injections specifically for delivery during a select portion of the cardiac cycle. In one example embodiment, one or more electrodes may be used as stimulation electrodes, e.g., to pace the heart during delivery. In this way, the cardiac cycle is made to be predictable and injection can be timed and synchronized to it. In fact, the beat-to-beat period can be artificially lengthened so as to permit complete delivery of microneedles during a specific (and relatively) stationary phase of the cardiac cycle. In one example embodiment, the delivery device includes one or more stimulation and/or sensing electrodes. In another example embodiment, sensors may be used to sense contractions of the heart, thereby allowing delivery to be timed with cardiac contractions. For example, it may be desirable to deliver microneedles between contractions of the heart.

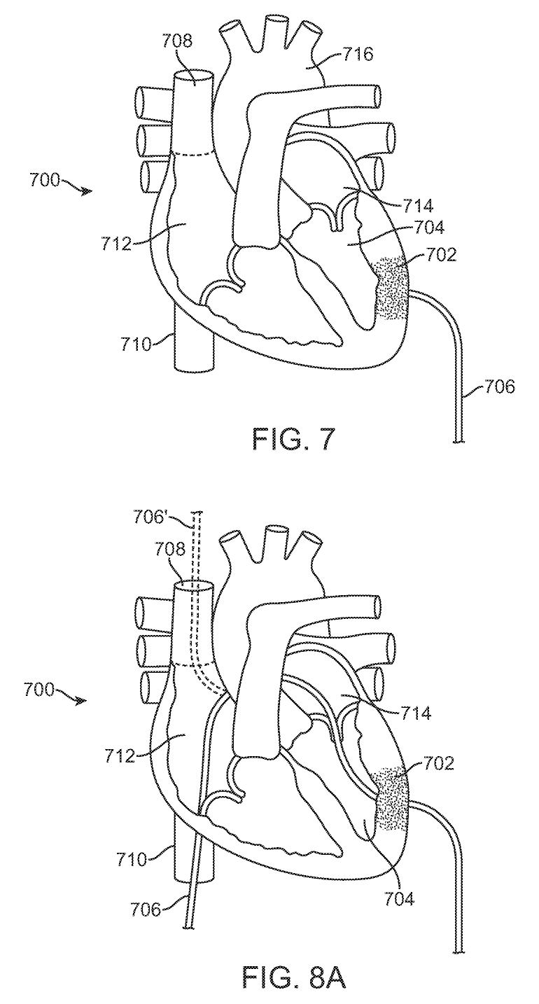

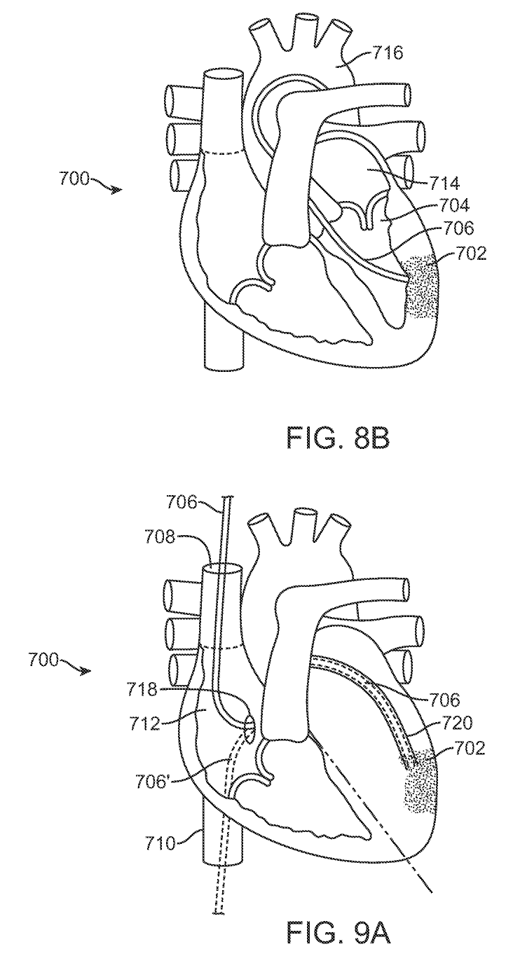

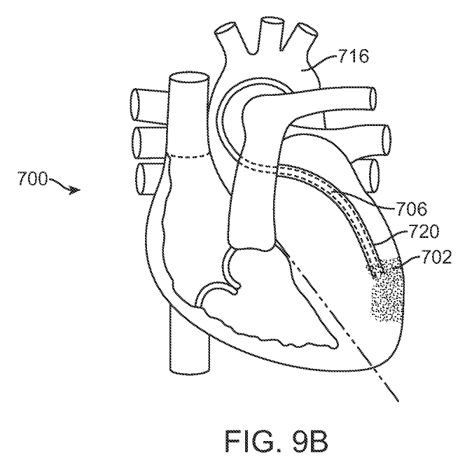

Microneedles, using the systems described herein, can also be delivered to the cardiac wall using a catheter system. Suitable catheter delivery systems include systems having multiple biaxial or coaxial lumens with staggered or flush tips. The catheter systems can include needles or other injection devices located at the distal end, and syringes at the proximal end. A catheter may be introduced endovascularly into a blood vessel until the distal portion is adjacent the desired treatment location. When catheter systems are used, a clinician can navigate to a patient's heart using one of the plurality of routes known for accessing the heart through the vasculature, or navigation to a heart chamber for delivery of the compositions epicardially (FIG. 7), endocardially (FIG. 8A-B) or transvascularly (FIG. 9A-B). In FIGS. 7 and 8 the entire heart is shown in cross section. In FIG. 9 the right ventricle and atrium are shown in cross-section while the left ventricle and atrium are shown closed with the epicardial surface and its coronary vessels in view

Epicardial delivery comprises accessing treatment site 702, in a non-limiting example, in left ventricle 704 of heart 700 from the epicardial, that is, exterior, surface of the heart as depicted in FIG. 7 and injecting one or more microneedles into treatment site 702 with delivery device 706.

Endocardial delivery (FIGS. 8A and 8B) comprises accessing treatment site 702, for example, in left ventricle 704 of heart 700, with delivery device 706, 706′ percutaneously through an anterograde approach (FIG. 8A) through superior vena cava 708 (delivery device 706′) or inferior vena cava 710 (delivery device 706) into right ventricle 712. Delivery device 706 is passed through the interatrial septum into left atrium 714 and then into left ventricle 704 to reach treatment site 702 where the microneedles and/or bioactive agents are injected with delivery device 706. An alternative endocardial delivery method depicted in FIG. 8B comprises accessing treatment site 702, for example, in left ventricle 704 of heart 700, with delivery device 706 percutaneously through a retrograde approach through aorta 716 into left atrium 714 and then into left ventricle 704 to reach treatment site 702 where the microneedles and/or bioactive agents are injected with delivery device 706.

Transvascular delivery of the microneedles and/or bioactive agents (FIGS. 9A and 9B) comprises accessing treatment site 702, for example, in left ventricle 704 of heart 700, with delivery device 706, 706′ percutaneously through a venous approach (FIG. 9A) through superior vena cava 708 (delivery device 706) or inferior vena cava 710 (delivery device 706′) into right ventricle 712. Delivery device 706 is passed through coronary sinus 718 into the cardiac venous system via these veins and, if needed, leaving these veins by tracking through cardiac tissue, it reaches treatment site 702 where the microneedles and/or bioactive agents are injected with delivery device 706. An alternative transvascular delivery method depicted in FIG. 9B comprises accessing a treatment site 702, for example, in left ventricle 704 of heart 700, with delivery device 706 percutaneously through an arterial approach through aorta 716 into coronary artery 720 to reach treatment site 702 where the microneedles and/or bioactive agents are injected with delivery device 706.

Devices for injecting the microneedles and/or bioactive agents can include refrigerated parts for keeping the various components cool. Various embodiments can include refrigerated/cooled chambers and/or an agitator mechanism. The devices can include heating or cooling components used to heat or cool the cardiac tissue or microneedles. Some devices can include catheters or other delivery devices with a cooled lumen or lumens for keeping components of the injected microneedles cool while they are traveling to their destination. This cooling component may be particularly important when live stem cells are being delivered using the microneedles described herein.

If a clinician is practicing methods described herein using a minimally invasive or percutaneous technique, he/she may need some sort of real-time visualization or navigation to ensure site-specific injections. Thus, at least one embodiment uses MNav technologies to superimpose pre-operative magnetic resonance imaging (MRI) or computed tomography (CT) images onto fluoroscopic images of a delivery catheter to track it in real-time to target sites. In one embodiment, the clinician uses a contrast agent and/or navigation technologies to track the needle-tip during injection in a virtual three-dimensional environment. This technique marks previous injections to ensure proper spacing of future injections.

The systems described herein may further include a feedback element or sensor for measuring a physiological condition to guide delivery of microneedles to a desired location. For example, an electrocardiogram (EKG) lead may be included on the distal tip or otherwise delivered within the selected tissue region to detect and guide injection towards electrically silent or quiet areas of cardiac tissue, or to allow electrical events within the heart to be monitored during delivery. During treatment, for example, the microneedles may be delivered into a tissue region until a desired condition is met. Also, local EKG monitoring can be used to target and guide injection towards electrically silent or quiet areas of cardiac tissue.

Further, a clinician may have the need for precise local placement and depth-control for each injection. In one example embodiment, the microneedles are injected to a depth in the cardiac wall that is approximately midway between the outside wall and the inside wall. In other embodiments, the microneedles are delivered to a depth that is closer to either the inside wall or the outside wall. The microneedles may be delivered intra-myocardially, sub-endocardially, or sub-epicardially. In another embodiment, the depth will vary based on the thickness of the target tissue and the depth is less at the apex of a heart than it is at other locations on the heart.

To achieve depth control, the systems include a stopper fixed (or adjustably fixed) on the needle shaft, at a desired distance from needle's distal tip, to prevent penetration into tissue beyond a specified depth. In at least one embodiment, the needle can be positioned to inject at an angle perpendicular (90 degrees) to the tissue, tangential (0 degrees) to the tissue, or any desired angle in between. Suction can facilitate controlled positioning and entry of the injector. Generally, the needle is injected perpendicular to the tissues, and therefore, the microneedles, if injected in a perpendicular plane relative to the needle are substantially parallel to the tissue surface. Such a configuration allows for more of an area to be treated than a simple, single injection alone.

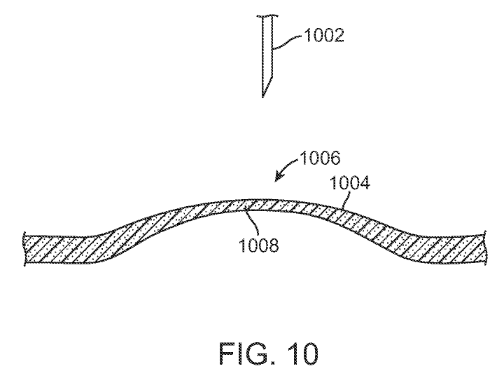

Referring now to FIG. 10, there can be seen an example of an injection wherein needle 1002 of a delivery device (not shown) is approaching at an angle generally perpendicular to cardiac wall 1004. Needle 1002 will puncture cardiac wall 1004 at point 1006 directly above desired delivery location 1008 within the cardiac tissue. The device may include one or more means, for example, as described above, to ensure that the needle achieves the desired penetration depth into the myocardium.

At least one embodiment uses a “Smart-Needle” to detect distance from the needle tip to the ventricular blood compartment or endocardial surface, so that the needle tip is maintained in the cardiac wall. Such a needle can rely on imaging around or ahead of the needle tip by imaging modes such as ultrasound.

At least one embodiment prevents backbleed out of the needle track, during and after removal of the needle, by keeping the needle in place for several seconds (e.g. 5-30 sec beyond the expected clotting time) following injection. Further, the systems can include a channel in the needle to inject a ‘plug’ of polymerizable substance which when formed or gelled prevents back-bleed, before removing needle. In at least one embodiment, the needle is left in place for the expected gelling time of the injected substance and then withdrawn. In one embodiment, the gelling time is about five seconds.

Some example embodiments can include sensors and other means to assist in directing the delivery device to a desired location, ensuring that the injections occur at a desired depth, ensuring the delivery device is at the treatment site, and other functions that may require some type of sensor or imaging means to be used. For example, real-time recording of electrical activity (e.g., EKG), pH, oxygenation, metabolites such as lactic acid, CO₂, or other local indicators of cardiac tissue viability or activity can be used to help guide the injections to the desired location. In some embodiments, the delivery device may include one or more sensors. For example, the sensors may be one or more electrical sensors, fiber optic sensors, chemical sensors, imaging sensors, structural sensors and/or proximity sensors that measure conductance. In one embodiment, the sensors may be tissue depth sensors for determining the depth of tissue adjacent the delivery device. In one embodiment, a sensor that detects pH, oxygenation, a blood metabolite, a tissue metabolite, etc. may be used at the end of the delivery device to alert the user if and when the tip has entered the chamber blood. This would cause the operator to re-position the delivery instrument before delivering the microneedles to the tissue. The one or more depth sensors may be used to control the depth of needle penetration into the tissue. In this way, the needle penetration depth can be controlled, for example, according to the thickness of tissue, e.g., tissue of a heart chamber wall. In some embodiments, sensors may be positioned or located on one or more needles of the delivery device. In some embodiments, sensors may be positioned or located on one or more tissue-contacting surfaces of the delivery device. In other embodiments, the delivery device may include one or more indicators. For example, a variety of indicators, e.g., visual or audible, may be used to indicate to the physician that the desired tissue depth has been achieved.

Furthermore, the delivery device may comprise sensors to allow the surgeon or clinician to ensure the delivery device is within the heart wall rather than in the ventricle at the time of injection. Non-limiting examples of sensors which would allow determination of the location of the injector include, pressure sensors, pH sensors and sensors for dissolved gases, such as oxygen. An additional sensor that may be associated with the delivery devices suitable for use include sensors which indicate flow of blood such as a backflow port or a backflow lumen which would inform a surgeon or clinician that the needle portion of the delivery device is in an area which has blood flow rather than within a tissue.

In addition to catheter delivery of microneedles, they can also, in some example embodiments, be delivered to injured tissue using direct injection to that tissue. For example, if cardiac tissue is the treatment target, injection can be made through a patient's chest, directly into the cardiac tissue. Depth measurement can be used to aid injecting the microneedles at an appropriate depth.

The location of the delivery can vary based on the size and shape of the injured region of cardiac tissue. In at least one example embodiment, the microneedles are delivered only into the injured cardiac tissue, while in other embodiments the peri-injury zone around the injured region is treated, and, in at least one other example embodiment, the microneedles are delivered into only the healthy tissue that borders an injured region. In other embodiments, the microneedles may be delivered to any combination of the regions of injured cardiac tissue, tissue in the peri-injury zone, and healthy tissue.

The timing of microneedle delivery relative to an injurious event will be based on the severity of the injury, the extent of the injury, the condition of the patient, and the progression of any tissue remodeling. In at least one example embodiment, delivery occurs within one to eight hours following an injurious event such as a myocardial infarction, for example within one to eight hours following ischemia-reperfusion (in the catheterization lab setting immediately after re-perfusion). In another example embodiment, delivery occurs within one hour of an injurious event. In yet another example embodiment, delivery occurs within three to four days after an injury (after clinical stabilization of the patient, which would make it safe for the patient to undergo a separate procedure). In at least one example embodiment delivery occurs more than one week after the injury, including up to months or years after injury. Other times for injecting microneedles and/or bioactive agents into the cardiac wall are also contemplated, including prior to any injurious event, and immediately upon finding an area of injured cardiac tissue (for preventing additional remodeling in older injuries). In another example embodiment, microneedles can be injected into the cardiac tissue years after an injurious event. In still another example embodiment, the microneedles are injected into the cardiac tissue from about 1 hour to about 2 years after an injurious event, from about 6 hours to about 1 year after an injurious event, from about 12 hours to about 9 months after an injurious event, from about 24 hours to about 6 months after an injurious event, or from about 48 hours to about 3 months after an injurious event. In yet another example embodiment, the microneedles and/or bioactive agents are injected into the cardiac tissue up to about 10 years after an injurious event.

In addition, it will be apparent to those skilled in the art that other injured tissues, in addition to injured cardiac tissue, would benefit from the systems and methods described herein. Examples of such tissues include ischemic tissues in organs or sites including, but not limited to, wounds, gastrointestinal tissue, kidney, liver, skin, and neural tissue such as brain, spinal cord and nerves.

### EXAMPLE 1

### Treatment of Injured Myocardium Using Catheter

A 76 year old man is recovering from a myocardial infarction 6 months prior. It is determined that there is damaged tissue on the left ventricle of his heart. The patient is prepped for stem cell treatment of the injured cardiac tissue. An eight non-biodegradable microneedle device is used in conjunction with a catheter delivery system. The catheter is deployed through the superior vena cava into the right ventricle. Then, the catheter is passed through the interatrial septum into the left atrium and then into the left ventricle to reach the injured cardiac tissue where the microneedles are injected. The injection results in two single rows of stem cells per injection track, the injection tracks resulting in a radial pattern each 45 degrees apart.

Upon follow-up examination six months later, the patient notices an increased ability to perform everyday tasks without becoming winded. Further, it is determined that the injured cardiac tissue has healed.

### EXAMPLE 2

### Treatment of Injured Myocardium Using Direct Injection

A 65 year old woman is recovering from a myocardial infarction four days prior. It is determined that there is damaged tissue on the left ventricle of her heart. The patient is prepped for stem cell treatment of the injured cardiac tissue. An eight non-biodegradable microneedle device is used in conjunction with a direct injection system. The needle is injected directly into the chest of the patient until the needle reaches the injured cardiac tissue and is at an appropriate depth. Then, the microneedles are injected. The injection results in two single rows of stem cells per injection track, the injection tracks resulting in a radial pattern each 45 degrees apart.

Upon follow-up examination two weeks later, the patient notices a decrease in labored breathing. Further, it is determined that the injured cardiac tissue has substantially healed.

### EXAMPLE 3

### Treatment of Injured Myocardium Using Biodegradable Microneedles and Catheter Delivery

A 69 year old man is recovering from blunt trauma to cardiac tissue on the left ventricle of his heart after a severe car accident. It is, in fact, determined that there is damaged tissue on the left ventricle of his heart. The patient is prepped for treatment of the injured cardiac tissue. An eight biodegradable microneedle device is used in conjunction with a catheter delivery system. The microneedles are manufactured from poly(D,L-lactide) impregnated with pacitaxel. The catheter is deployed through the superior vena cava into the right ventricle. Then, the catheter is passed through the interatrial septum into the left atrium and then into the left ventricle to reach the injured cardiac tissue where the microneedles are injected. The injection results in biodegradable microneedles left behind in a radial pattern, each 45 degrees apart. Upon follow-up examination a week later, it is determined that there is a substantial reduction in swelling of the cardiac tissue in the area of injury.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A microneedle drug delivery device comprising:
a needle comprising a body, a proximal end and a distal end, wherein said distal end is closed and said proximal end is attached to a delivery means, and said body has at least one channel traversing interior of said body from said distal end to said proximal end, said at least one channel terminates at an exit port at said distal end of said needle; and
at least one microneedle housed within said at least one channel for delivery through said exit port of said at least one channel.

2. The microneedle drug delivery device according to claim 1 wherein said delivery body has four channels.

3. The microneedle drug delivery device according to claim 1 wherein said delivery body has eight channels.

4. The microneedle drug delivery device according to claim 1 wherein said at least one microneedle contains at least one bioactive agent.

5. The microneedle drug delivery device according to claim 4 wherein said at least one microneedle is non-biodegradable.

6. The microneedle drug delivery device according to claim 5 wherein said at least one microneedle is metal.

7. The microneedle drug delivery device according to claim 1 wherein said at least one microneedle is injected into a tissue from said at least one channel of said needle.

8. The microneedle drug delivery device according to claim 7 wherein said at least one microneedle is injected into said tissue substantially perpendicular to said needle from said at least one channel of said needle.

9. The microneedle drug delivery device according to claim 4 wherein said at least one microneedle comprises at least one biodegradable polymer.

10. The microneedle drug delivery device according to claim 9 wherein said at least one bioactive agent is dispersed within said at least one biodegradable polymer.

11. The microneedle drug delivery device according to claim 4 wherein said at least one bioactive agent is selected from the group consisting of anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) zotarolimus (ABT-578), somatic cells, stem cells and combinations thereof.

12. A method of delivering a microneedle comprising:
a) selecting a patient who is a candidate for local delivery of at least one bioactive agent to a tissue in need thereof;
b) providing the microneedle injection device of claim 1;
c) inserting said needle into said tissue;
d) advancing said at least one microneedle out of said at least one channel through said exit port and into said tissue;
e) retracting said needle from said tissue; and
f) delivering said one or more bioactive agents using said microneedle to said tissue.

13. The method according to claim 12 further comprising the step of retracting said at least one microneedle back through said exit port into said at least one channel.

14. The method according to claim 12 wherein said at least one bioactive agent is selected from the group consisting of anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) zotarolimus (ABT-578), somatic cells, stem cells and combinations thereof.

15. The method according to claim 12 wherein said at least one microneedle is non-biodegradable.

16. The method according to claim 12 wherein said at least one microneedle is biodegradable.

17. The method according to claim 16 wherein said at least one microneedle comprises at least one biodegradable polymer and said at least one bioactive agent is dispersed within said at least one biodegradable polymer.

18. The method according to claim 12 wherein said at least one microneedle is injected into said tissue substantially perpendicular from said at least one channel of said needle.

19. The method according to claim 18 wherein eight microneedles are injected in a radial pattern into said tissue substantially perpendicular to said at least one channel of said needle.

20. A microneedle drug delivery device comprising:
a needle comprising a body, a proximal end and a distal end, wherein said distal end is closed and said proximal end is attached to a delivery means, and said body has eight channels traversing the interior of said body from said distal end to said proximal end, said eight channels terminate at eight exit ports at said distal end of said needle; and
eight microneedles comprising at least one bioactive agent, each of said eight microneedles housed within one of said eight channels for delivery through said eight exit ports of said eight channels.
